(19) United States
(12) Patent Application Publication
(10) Pub. No.: US 2009/0291878 A1
(43) Pub. Date: Nov. 26, 2009

(54) MODULATORS OF PROTEIN PHOSPHATASE 2A HOLOENYME

(75) Inventors: Yigong Shi, Plainsboro, NJ (US); Yongna Xing, Plainsboro, NJ (US); Yanhui Xu, Princeton, NJ (US)

Correspondence Address: PEPPER HAMILTON LLP, ONE MELLON CENTER, 50TH FLOOR, 500 GRANT STREET, PITTSBURGH, PA, 15219, US

(73) Assignee: THE TRUSTEES OF THE UNIVERSITY OF PRINCETON, Princeton, NJ (US)

(21) Appl. No.: 12/447,089

(22) PCT Filed: Oct. 29, 2007
(86) PCT No.: PCT/US07/82833

§ 371 Date: Apr. 24, 2009

Related U.S. Application Data
(60) Provisional application No. 60/855,183, filed on Oct. 30, 2006.

(51) Int. Cl. A61K 38/00; C07K 2/00; G06G 7/48

(52) U.S. Cl. 514/2; 530/300; 703/12

(57) ABSTRACT

Atomic coordinates for human serine/threonine protein phosphotase 2A (PP2A) holoenzyme, as well as methods for using these atomic coordinates to prepare inhibitors of PP2A and inhibitors prepared using such methods are provided herein. A biochemical analysis of the interactions of PP2A holoenzyme is also provided. Compositions including mimetics and small molecules of the invention and, optionally, secondary agents may be used to treat disorders in which PP2A activity plays a contributing role.

### CROSS-REFERENCE TO RELATED APPLICATIONS

This application claims priority to and benefit of U.S. Provisional Application No. 60/855,183, entitled “Structure of the Protein Phosphatase 2A Holoenzyme”, filed on Oct. 30, 2006; the entire contents of which are hereby incorporated by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

The invention presented herein provides compositions and methods for modulation of protein phosphatase 2A holoenzyme.

2. Description of Related Art

Reversible protein phosphorylation is a fundamental regulatory mechanism in all aspects of biology. Protein phosphatase 2A (PP2A) is a major serine/threonine phosphatase involved in many aspects of cellular function including, for example, cell cycle regulation, cell growth control, development, regulation of multiple signal transduction pathways, cytoskeleton dynamics, and cell mobility. Additionally, PP2A is also an important tumor suppressor protein.

PP2A holoenzyme is made up of at least three subunits (FIG. 1A). The PP2A core is made up of a 36 kDa catalytic (C) subunit and a 65 kDa scaffold (A) subunit. The C- and A-subunits each have two isoforms in mammalian cells, α and β, which share significant sequence similarity. Although in both cases, the α isoform is more abundant than the β isoform. PP2A core interacts with a third regulatory (B) subunit to form an active hetero-trimeric holoenzyme. B-subunits have been separated into four subfamilies: B (or PR55), B′ (or B56or PR61), B″ (or PR72), and B′″ (or PR93 or PR110), with at least 16 members in each subfamily. Among the four subfamilies, sequence similarity is very low, and the expression level of various types of B-subunits has been shown to be highly diverse depending upon cell types and tissues. The B-subunit of the heterotrimeric PP2A holoenzyme may determine the substrate specificity as well as the spatial and temporal functions of PP2A. For example, the PP2A holoenzyme involving the B′-subunit family may play an essential role in cell cycle progression, through direct interaction with the protein Shugoshin.

Inactivation of both the α and β isoforms of the PP2A holoenzyme A-subunit has been linked to several forms of cancer. For example, mutations in the A-subunit, Glu64 mutated to Asp (E64D) and Glu64 to Gly (E64G), may result in compromised binding to the B-subunit or C-subunit or substantially reduce binding between the B- and C-subunits. Several missense mutations associated with human tumor types have been identified in the β isoform of the A-subunit including, for example, P65S, L101P, K343E, D504G, and V545A which correspond to P53S, L89P, K331E, D492G, and V533A, respectively, in α isoform. In addition, an N-terminally truncated B-subunit mutant (B′γ1) has been shown to be associated with an increased metastasis in melanoma cells.

### BRIEF SUMMARY OF THE INVENTION

Various embodiments of the invention described herein are directed to a protein phosphatase 2A (PP2A) binding compound including a molecule having a three-dimensional structure corresponding to atomic coordinates derived from at least a portion of an atomic model of protein phosphatase 2A (PP2A) holoenzyme or protein phosphatase 2A (PP2A) holoenzyme bound to microcystin-LR.

In some embodiments, the compound may be an inhibitor of protein phosphatase 2A (PP2A). In other embodiments, the compound may have a three-dimensional structure corresponding to atomic coordinates of at least a portion microcystin-LR or a combination thereof bound to protein phosphatase 2A. (PP2A) holoenzyme and may make interactions with the catalytic (C) subunit of protein phosphatase 2A (PP2A) holoenzyme that correspond to at least a portion of the interactions observed between the catalytic (C) subunit of protein phosphatase 2A (PP2A) holoenzyme and microcystin-LR. In still other embodiments, the compound may bind protein phosphatase 2A (PP2A) at a binding site for microcystin-LR on the catalytic (C) subunit of PP2A.

Various embodiments also include a molecule having a shape, a charge, a size or combinations thereof substantially corresponding to a portion of protein phosphatase 2A (PP2A) holoenzyme, and various other embodiments include a molecule haying a shape, a charge, a size or combinations thereof substantially complementary to a portion of protein phosphatase 2A (PP2A) holoenzyme. In some embodiments, such a molecule may bind to a catalytic (C) subunit of protein phosphatase 2A (PP2A), a scaffolding (A) subunit of protein phosphatase 2A (PP2A) or a regulatory (B) subunit of protein phosphatase 2A (PP2A) at an interface between the catalytic (C) subunit and the scaffolding (A) subunit, a scaffolding (A) subunit and a regulatory (B) subunit, a catalytic (C) subunit and regulatory (B) subunit, or a combination thereof. In other embodiments, the molecule may be substantially complementary to a portion of a scaffolding (A) subunit of protein phosphatase 2A (PP2A) holoenzyme, and in other embodiments, the compound may bind a scaffolding (A) subunit of PP2A holoenzyme and inhibit flexibility of the scaffolding (A) subunit. Embodiments also include a molecule that may be substantially complementary to a portion of a regulatory (B) subunit of protein phosphatase 2A (PP2A) holoenzyme, and in some embodiments, such a molecule may inhibit access of substrate to the active site of protein phosphatase 2A (PP2A) holoenzyme or a molecule may inhibit formation of an active protein phosphatase 2A holoenzyme.

In certain embodiments, a compound as described herein may bind to at least a portion of protein phosphatase 2A (PP2A) holoenzyme with a greater affinity than a naturally occurring substrate, and in some embodiments, a molecule may inhibit protein phosphatase 2A (PP2A) catalyzed tyrosine phosphorylation, serine phosphorylation, threonine phosphorylation or a combination thereof. In still other embodiments, a molecule may deviates from the atomic coordinates of the at least a portion of PP2A holoenzyme by a root mean square deviation of less than about 10 angstroms, and in further embodiments, a molecule may deviate from the atomic coordinates of the at least a portion of PP2A holoenzyme by a root mean square deviation of less than about 2 angstroms.

Certain other embodiments in the compound and a pharmaceutically acceptable excipient or carrier.

Various other embodiments described herein in a method for preparing a PP2A modulating compound including the steps of applying a three-dimensional molecular modeling algorithm to the atomic coordinates of at least a portion of PP2A holoenzyme; determining spatial coordinates of the at least a portion of PP2A holoenzyme; electronically screening stored spatial coordinates of candidate compounds against the spatial coordinates of the at least a portion of PP2A holoenzyme; identifying a compound that is substantially similar to the at least a portion of PP2A holoenzyme; and synthesizing the identified compound.

In some embodiments, the method may also include the step of identifying a candidate compound that deviates from the atomic coordinates of the at least a portion of PP2A holoenzyme by a root mean square deviation of less than about 10 angstroms, and in other embodiments, the method may include the step of testing the identified compound for binding at least a portion of PP2A. Still other embodiments, may include the step of testing the identified compound for inhibiting PP2A activity and, in certain other, the step of testing the identified compound inhibits tyrosine phosphorylation, serine phosphorylation, threonine phosphorylation or a combination thereof catalyzed by PP2A holoenzyme.

In certain embodiments, the step of electronically screening stored spatial coordinates may include the step of identifying a compound that has a shape, a charge distribution, a size or a combination thereof substantially similar to a portion of PP2A holoenzyme.

In various other embodiments, the portion of the spatial coordinates of the PP2A holoenzyme may include an interface between any one of: scaffolding (A) subunit and catalytic (C) subunit, scaffolding (A) subunit and regulatory (B) subunit and regulatory (B) subunit and catalytic (C) subunit. In some embodiments, the identified compound may interrupt the interface and inhibits PP2A holoenzyme assembly.

Still other embodiments include a method for preparing a PP2A inhibitor including the steps of: applying a three-dimensional molecular modeling algorithm to the atomic coordinates of PP2A holoenzyme; determining spatial coordinates of a portion of PP2A holoenzyme corresponding to a concave surface of a regulatory (B) subunit; electronically screening stored spatial coordinates of candidate compounds against the spatial coordinates of the at least a portion of PP2A holoenzyme corresponding to the concave surface of the regulatory (B) subunit; identifying a compound that is substantially complementary to the concave surface of PP2A holoenzyme regulatory (B) subunit; and synthesizing the identified compound.

In some embodiment, the method may include the step of identifying a compound that has a shape, a charge distribution, a size or a combination thereof substantially complementary to the concave surface of PP2A holoenzyme regulatory (B) subunit. In other embodiment, the compound may include a plurality of basic moieties, and in still other embodiments, the identified compound inhibits entry of substrate into an active site of PP2A catalytic (C) subunit.

In certain embodiments, the method may also include the steps of: identifying one or more PP2A substrate proteins; isolating at least a portion of the one or more PP2A substrate proteins where PP2A holoenzyme is likely to bind the one or more PP2A substrate proteins; determining spatial coordinates of the at least a portion of the one or more PP2A substrate proteins; and identifying a compound that is substantially similar to the at least a portion of the one or more PP2A substrate proteins. In certain other embodiments, the step of isolating one or more PP2A substrate proteins may further include the steps of: identifying more than one PP2A substrate proteins; performing an alignment of the more than one PP2A substrate proteins; and isolating at least a portion of the more than one PP2A substrate proteins that share sequence similarity or secondary structure similarity.

In further embodiments, the method may include the step of testing the identified compound for binding to PP2A holoenzyme.

Various other embodiments are directed to a pharmaceutical composition comprising: an effective amount of a compound having a three-dimensional structure corresponding to atomic coordinates of at least a portion of PP2A; and a pharmaceutically acceptable excipient or carrier. In some embodiments, the pharmaceutical composition may include a compound binds to PP2A holoenzyme.

Embodiments of the invention described herein further include a system for identifying PP2A modulators including: a processor and a processor readable storage medium in communication with the processor readable storage medium comprising the atomic coordinates of at least a portion of PP2A holoenzyme. In some embodiments, the processor readable storage medium may further include one or more programming instructions for: applying a three-dimensional modeling algorithm to the atomic coordinates of PP2A holoenzyme; determining spatial coordinates of at least a portion of the PP2A holoenzyme; electronically screening spatial coordinates of candidate compounds with the spatial coordinates of the at least a portion of the PP2A holoenzyme; and identifying a candidate compound whose spatial coordinates are substantially similar to the spatial coordinates of the at least a portion of the PP2A holoenzyme; or identifying a candidate compound whose spatial coordinates are substantially complementary to the spatial coordinates of the at least a portion of the PP2A holoenzyme.

In some embodiments, the one or more programming instructions for identifying a candidate compound whose spatial coordinates are substantially similar to the spatial coordinates of the at least a portion of the PP2A holoenzyme may include one or more programming instructions for identifying a compound that deviates from the spatial coordinates of the at least a portion of the PP2A holoenzyme by a user defined threshold, and in others the one or more programming instructions for identifying a compound whose spatial coordinates are substantially similar to the at least a portion of the PP2A holoenzyme comprise one or more programming instructions for identifying a compound having one or more of: a size within a user defined threshold; a charge within a user defined threshold; or a shape with a user defined threshold.

In still other embodiments, the one or more programming instructions for electronically screening spatial coordinates of a candidate compound comprises one or more programming instructions for simulating binding of the candidate compound to the PP2A holoenzyme.

In certain embodiments, the system may include an output device in communication with the processor. In certain other embodiments, the processor readable storage medium may further include one or more programming instructions for: applying a three-dimensional modeling algorithm to the atomic coordinates of PP2A holoenzyme; determining spatial coordinates of at least a portion of the PP2A holoenzyme; generating a visual signal arid relaying the visual signal to the output device; and electronically designing a compound that is substantially similar to the at least a portion of the PP2A holoenzyme; or electronically designing a compound that is substantially complementary to the at least a portion of the PP2A holoenzyme.

### DESCRIPTION OF DRAWINGS

For a fuller understanding of the nature and advantages of the present invention, reference should be made to the following detailed description taken in connection with the accompanying drawings. The file of this patent contains at least one drawing/photograph executed in color. Copies of this patent with color drawing(s)/photograph(s) will be provided to the USPTO upon request and payment of the necessary fee. All figures where structural representations are shown were prepared using MOLSCRIPT (Kraulis (1991) J Appl Crystallogr 24:946-950) and GRASP (Nicholls et al. (1991) Proteins: Struct Funct Genet 11:281-296).

FIG. 1A shows a three-dimensional molecular model of PP2A holoenzyme: the catalytic (C) subunit is blue, the scaffolding (A) subunit is green, and the regulatory (B) subunit is yellow.

FIG. 1B shows a molecular model of PP2A holoenzyme: the C-subunit is a wire diagram, the A-subunit (front) and B-subunit (back) are surface representations.

FIG. 2A shows a surface representation of the B-subunit including surface charges.

FIG. 2B shows the B-subunit (yellow) superimposed over the A-subunit.

FIG. 2C is a stereo diagram of the HEAT-like repeats 1-8 of the B-subunit,

FIG. 3 is an alignment of B′-subunits with secondary structural elements provided above the alignment and residues that appear to interact with the A-subunit and B-subunit indicated with filled circles.

FIG. 4A shows the PP2A holoenzyme: the C-subunit is a wire diagram, the A-subunit (front) and B-subunit (back) are surface representations. Area 1 and Area 2 are circled.

FIG. 4B shows a portion of the interface in Area 1.

FIG. 4C shows a portion of the interface in Area 2.

FIG. 4D shows a portion of an extended loop of HEAT-like repeat 2 of the B-subunit that appear to interact with the C-subunit.

FIG. 4E shows PP2A holoenzyme: the B-subunit is a wire diagram, the C-subunit (front) and A-subunit (back) are surface representations.

FIG. 4F shows a portion of an interface between the HEAT-like repeats 4 and 5 of the B-subunit and HEAT repeats 2-5 of the A-subunit.

FIG. 5A shows SDS-PAGE of the results of a GST-mediated pull down assay of B-subunit with various mutant A-subunits.

FIG. 5B shows SDS-PAGE of the results of a GST-mediated pull down assay of C-subunit with various mutant A-subunits.

FIG. 6A shows an overlay of PP2A core and PP2A holoenzyme.

FIG. 6B shows an overlay of the A-subunit of PP2A core and PP2A holoenzyme. The C-terminal 5 HEAT repeats are aligned in the left panel and the N-terminal 10 HEAT repeats are aligned in the left panel. The switch point is circled.

FIG. 6C shows an overlay of HEAT repeats 10, 11 and 12 of PP2A core and PP2A holoenzyme.

FIG. 6D shows a stereo diagram overlay of a portion of HEAT repeat 11 from PP2A core and PP2A holoenzyme encompassing the switch point.

FIG. 7A shows representative data from a peak fractions of gel-filtration separation of methylated, unmethylated and truncated C-subunit holoenzyme.

FIG. 7B shows a molecular model of PP2A holoenzyme prepared from truncated C-subunit.

### DETAILED DESCRIPTION

It must be noted that, as used herein and in the appended claims, the singular forms “a,” “an,” and “the” include plural reference unless the context clearly dictates otherwise. Unless defined otherwise, all technical and scientific terms used herein, have the same meanings as commonly understood by one of ordinary skill in the art. Although any methods similar or equivalent to those described, herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The terms “mimetic,” “peptide mimetic,” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically produced peptides, recombinantly or chemically modified peptides, as well as non-peptide agents, such as small molecule drug mimetics as further described below. Mimetic compounds can have additional characteristics that enhance their therapeutic application, such as increased cell permeability, greater affinity and/or avidity, and prolonged biological half-life.

As used herein, the terms “pharmaceutically acceptable,” “physiologically tolerable,” and grammatical variations thereof, as they refer to compositions, carriers, diluents, and reagents, are used interchangeably and represent that the materials are capable of administration upon a mammal without the production of undesirable physiological effects such as nausea, dizziness, rash, or gastric upset.

“Providing,” when used in conjunction with a therapeutic, means to administer a therapeutic directly into or onto a target tissue, or to administer a therapeutic to a patient whereby the therapeutic positively impacts the tissue to which it is targeted.

As used herein, “subject,” “patient” or “individual” refers to an animal or mammal including, but not limited to, a human, dog, cat, horse, cow, pig, sheep, goat, chicken, monkey, rabbit, rat, or mouse, etc.

As used herein, the term “therapeutic” means an agent utilized to treat, combat, ameliorate, prevent or improve an unwanted condition or disease of a patient. Embodiments of the present invention are directed, to promote apoptosis and thus, cell death.

The terms “therapeutically effective amount” or “effective amount,” as used herein, may be used interchangeably arid refer to an amount of a therapeutic compound component of the present invention. For example, a therapeutically effective amount of a therapeutic compound is a predetermined amount calculated to achieve the desired effect, i.e., to effectively modulate the activity of protein phosphatase 2A (PP2A).

“Inhibitor” means a compound which reduces or prevents a particular interaction or reaction. For example, an inhibitor may bind to PP2A C-subunit inactivating the C-subunit and inhibiting the phosphotyrosyl activity of PP2A.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable and formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

The invention described herein is generally directed to atomic coordinates defining PP2A holoenzyme, methods for using the atomic coordinates of PP2A holoenzyme, mimetics and small molecules prepared using such methods, and pharmaceutical compositions made from mimetics and small molecules so prepared.

The atomic coordinates of PP2A holoenzyme were determined by preparing a holoenzyme of full-length human catalytic (C) subunit and trypsin-digested catalytic (C(T)) subunit (residues 1-294) in which the C-terminal 15 amino acids have been removed, full-length human scaffolding (A) subunit and full-length human regulatory (B) subunit. Preparation of PP2A holoenzyme may begin by assembling a PP2A core of the C- and A-subunits. For example, the PP2A core may be assembled and isolated as described in PCT Patent Application No. PCT/US2007/81260, entitled “Modulators of Protein Phosphatase 2A”, hereby incorporated by reference in its entirety. The PP2A core may then be methylated using, for example, PP2A-specific leucine carboxyl methyltransferase (LCMT) and S-adenosyl methionine (SAM). The holoenzyme may be formed by combining methylated PP2A core with a stoichiometric amount of B-subunit and then purifying the resulting PP2A holoenzyme to homogeneity using, for example, gel filtration chromatography.

Crystals of PP2A holoenzyme may be prepared by any method. In one embodiment, PP2A holoenzyme may be incubated with a co-agent, such as, for example, microcystin-LR (MCLR), to facilitate crystallization. In another embodiment, small crystals of the PP2A holoenzyme may be obtained using hanging-drop vapor-diffusion and larger diffraction-quality crystals may be generated by macro-seeding. Crystals prepared by this method were found to diffract X-rays to about 3.3 Å resolution using synchrotron source.

The diffraction data generated using crystals prepared as described above may be used to determine atomic coordinates for PP2A holoenzyme using any method known in the art, and such atomic coordinates may be used to construct an atomic model of PP2A. holoenzyme. For example, atomic coordinates of PP2A holoenzyme may be determined from crystallographic diffraction data collected using a combination of molecular replacement and single-wavelength anomalous dispersion. The diffraction and structural data presented herein include atomic models for three PP2A holoenzyme species prepared using such methods: native PP2A holoenzyme, PP2A holoenzyme formed from selenomethionine-substituted C(T)-subunit and A-subunit containing PP2A core with B′-γ1 B-subunit and PP2A holoenzyme formed from selenomethionine-substituted C(T)-subunit and A-subunit containing PP2A core B′-γ3 B-subunit. All of the structures were determined to satisfactory resolution. For example, the atomic model of the native, holoenzyme prepared in this way was refined to about 3.3 Å resolution. The statistical analysis of the crystallographic data acquired for each of these, holoenzyme species is provided in Table 1.

TABLE 1

| | | |
|---|---|---|
| Native | SeMet (A & C) | SeMet (A & C) |

Crystallographic Data Collection Statistics

| | | | |
|---|---|---|---|
| Protein components | A, B, C | A, B′ - γ3, C(T) | A, B′ - γ1, C(T) |
| Space group | P212121 | P212121 | P212121 |
| Resolution (outer shell) (Å) | 100-3.3 (3.42-3.30) | 100-3.6 (3.73-3.60) | 100-3.8 (3.94-3.80) |
| Total observations | 443,360 | 912,830 | 261,894 |
| Unique observations | 70,289 | 53,992 | 45,951 |
| Data coverage (outer shell) | 99.9% (99.9%) | 99.9% (100.0%) | 99.7% (99.9%) |
| R_{sym} (outer shell) | 0.104 (0.444) | 0.116 (0.446) | 0.109 (0.432) |

Refinement

| | | | |
|---|---|---|---|
| Resolution (outer shell) (Å) | 100-3.3 (3.42-3.30) | 100-3.6 (3.73-3.60) | 100-3.8 (3.94-3.80) |
| Number of reflections (\|F\| > 0) | 70,199 | 53,472 | 46,983 |
| Data coverage | 98.4% | 97.4% | 99.6% |
| R_{work} (outer shell) | 0.252 (0.392) | 0.267 (0.364) | 0.282 (0.332) |
| R_{free} (outer shell) | 0.297 (0.434) | 0.331 (0.439) | 0.335 (0.418) |
| Total number of atoms | 20,474 | 20,212 | 20,212 |
| Number of waters | 0 | 0 | 0 |
| R.m.s.d. bond length (Å) | 0.0083 | 0.010 | 0.011 |
| R.m.s.d. bond angles (degree) | 1.38 | 1.53 | 1.56 |

Average B factor

| | | | |
|---|---|---|---|
| Aα - two copies per a.u. | 69.6, 60.1 | 70.3, 48.1 | 81.3, 67.3 |
| Cα - two copies per a.u. | 72.7, 70.1 | 69.2, 59.5 | 80.3, 76.4 |
| B′- γ - two copies per a.u. | 85.8, 79.1 | 84.3, 70.5 | 95.2, 86.3 |

Ramachandran Plot

| | | | |
|---|---|---|---|
| Most favored (%) | 80.6 | 75.3 | 73.2 |
| Additionally allowed (%) | 17.6 | 22.5 | 23.8 |
| Generously allowed (%) | 1.4 | 2.0 | 2.5 |
| Disallowed (%) | 0.3 | 0.3 | 0.4 |

R_{sym} = ΣₕΣᵢ|I_{h,i} − Iₕ|/ΣₕΣᵢI_{h,i}, where Iₕ is the mean intensity of the i observations of symmetry related reflections of h. R = Σ|F_{obs} − F_{calc}|/ΣF_{obs}, where F_{obs} = Fₚ, and F_{calc} is the calculated protein structure factor from the atomic model (R_{free} was calculated with 5% of the reflections). R.m.s.d. in bond lengths and angles are the deviations from ideal values.

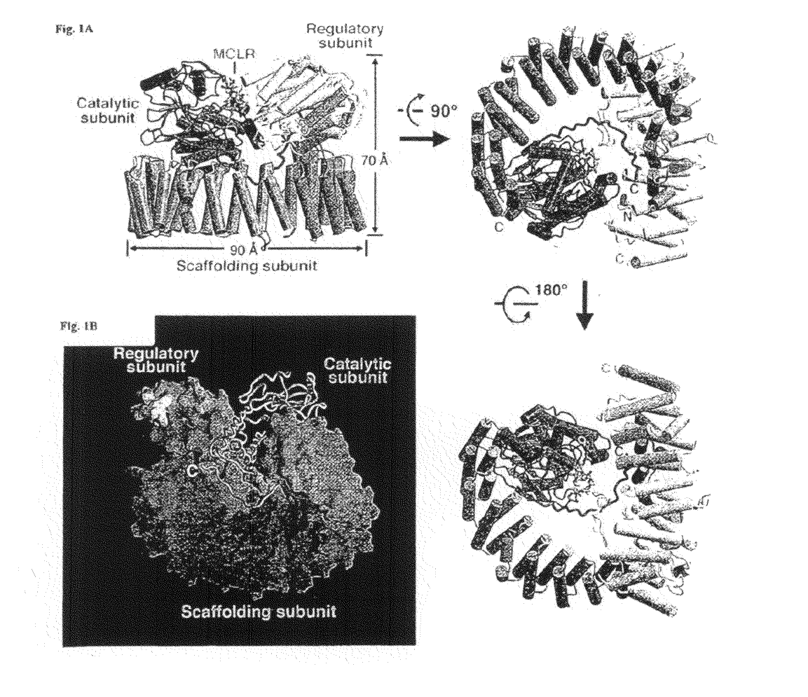

Based on the atomic models, prepared as described above, PP2A holoenzyme appears to exhibit a compact architecture, measuring about 90 Å×90 Å×70 Å. As illustrated in FIG. 1, the B′-subunit appears to make extensive interactions with both the A-subunit and the C-subunit. In total, about 4,300 Å² of solvent accessible surface area may be buried as a result of interaction between the B-subunit and PP2A core, and approximately 55 percent of the total buried surface area appears to be as a result of interactions between B-subunit and C-subunit.

As illustrated in FIG. 1, the A-subunit, shown in green, has a C-shaped structure including 15 HEAT repeats, which are characterized by repeating, double-layered, antiparallel α-helices. The inter-helical region within each HEAT repeat forms a contiguous ridge (“the ridge”). The interaction between A- and C-subunits of PP2A holoenzyme appears to result in the burial of about 2,070 Å² of exposed surface area. Compared to the free A-subunit and A-subunit of PP2A core enzyme, the A-subunit of PP2A holoenzyme appears to have undergone significant structural rearrangements. For example, the C-subunit of PP2A holoenzyme, shown in blue, appears to bind to the C-terminal end of the A-subunit through interactions with the ridge of HEAT repeats 11-15, and this interaction is characterized by a “kink” in the A-subunit between HEAT repeats 12 and 13 which result in a conformation change in the region encompassing HEAT repeats 11-15.

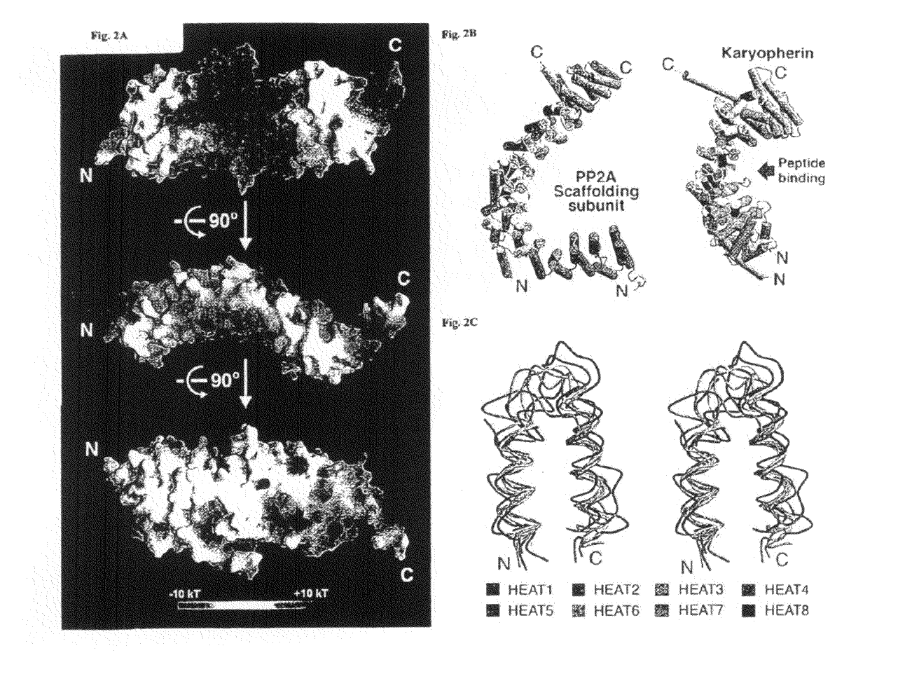

As illustrated in FIG. 1A and FIG. 2, the B-subunit of PP2A holoenzyme, shown in yellow, appears to have an elongated, super-helical structure with an apparent curvature. The B-subunit appears to include 18 α-helices stacked against each other laterally to create eight HEAT-like repeats that, despite having very little sequence homology, closely resemble the HEAT repeats of the A-subunit. FIG. 2B, left panel illustrates the structural similarity of the B-subunit HEAT-like repeats and the A-subunit HEAT repeats by showing an overlay of the B- and A-subunits which result in a root-mean-squared deviation (rmsd) of about 5.1 Å over 254 aligned backbone alpha carbon atoms that make up the eight HEAT-like repeats in B-subunit. Of the B-subunit HEAT-like repeats, repeat seven appears to be most structurally homologous to the HEAT repeats of the A-subunit. The eight HEAT-like repeats of the B-subunit appear to exhibit little sequence homology among themselves and lack a strong consensus sequence. However, as shown in FIG. 2C, each of the eight HEAT-like repeats can be superimposed with each other with a pair-wise rmsd 0.9-2.5 Å.

FIG. 2A is a surface model of the B-subunit including apparent surface charge over the entire surface of the B-subunit. As can be observed in FIG. 2A, the concave side of the B-subunit appears enriched with negatively charged amino acids, whereas the convex side appears to contain a number of hydrophobic residues. The hydrophobic residues on the convex side of the B-subunit may make contributions, in binding to the A-subunit as evidenced by the apparent interaction between the A- and B-subunits as illustrated, in FIG. 1. Without wishing to be bound by theory, the negatively charged amino acids of the concave side of B-subunit may be involved in contacting substrate proteins. For example, the arrangement of the C-, A-, and B-subunits appears to leave the highly acidic, concave side of the B-subunit unoccupied as indicated by FIG. 1A. Moreover, the concave surface appears to tilt toward the active site pocket of the C-subunit. Additionally, a systematic structure-based search of the Protein Data Bank using the DAL1 server identified a number of close structural homologs of the B-subunit including, among others, a nuclear import factor, karyopherin α, the armadillo repeat protein, β-catenin, and PP2A A-subunit, as indicated in FIG. 2B which shows an overlay of B-subunit (yellow) with PP2A A-subunit (gray, left panel) and karyopherin α (gray, right panel). Of these, karyopherin α and β-catenin have been shown to interact with peptides on their concave surfaces.

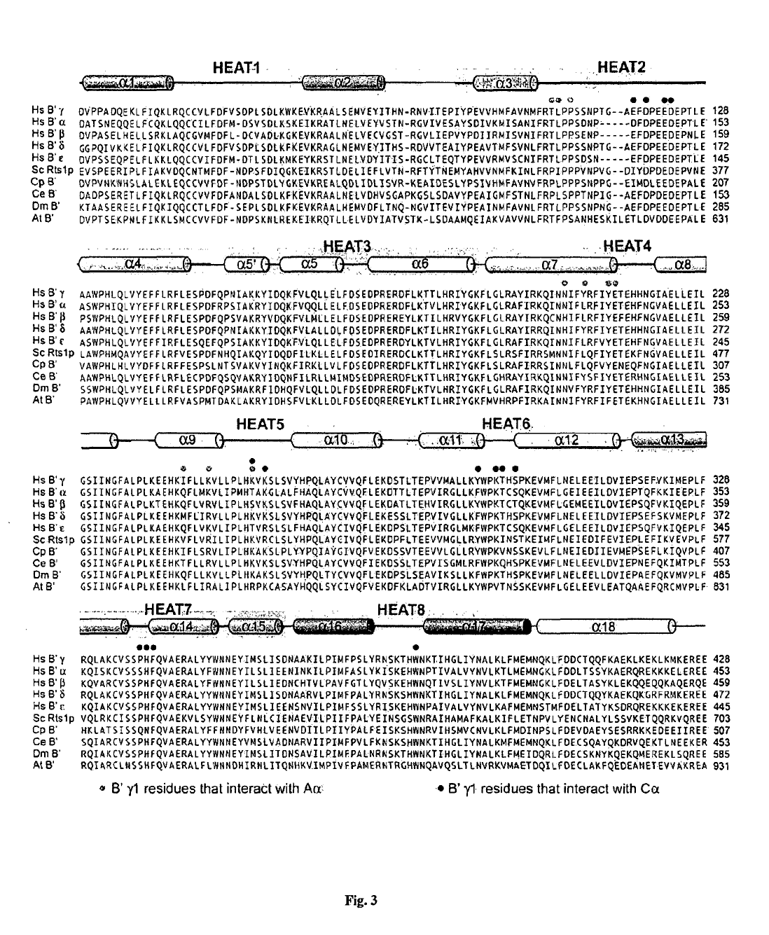

PP2A B-subunits appear to share sequence homology across species. For example, FIG. 3 shows a sequence alignment of five B-subunit isoforms: human B′-γ1 (Hs B′γ), human B′-α1 (Hs B′α), human B′-β1 (Hs B′β), human B′-δ1 (Hs B′δ), human B′-ε1 (Hs B′ε), Saccharomyces cerevisiae Rts1p (Sc Rts1p), Chizosaccharomyces pombe B′ (Cp B′), Caenorhabitis elegans B′ (Ce B′), Drosphila melanogaster (Dm B′), and Arabidopsis thalia (At B′). Conserved residues are highlighted in yellow. Secondary structural elements of the atomic model for human PP2A B-subunit are provided above the sequence alignment and each HEAT-like repeat is labeled and shaded in a different color. Amino acid residues that interact with the A-subunit and/or the C-subunit are identified by green or blue circles, respectively, above the sequence. These data suggest strong sequence homology among diverse species. In particular, amino acids that appear to be involved in contacting the A- and C-subunits and amino acid residues in close proximity to these residues appear to be highly conserved.

The B-subunit appears to bind to the ridge of HEAT repeats 2-8 in A-subunit and may directly interact with at least three surfaces of C-subunit, including, but not limited to, helix α-5 and the C-terminal loop of the C-subunit. As illustrated in FIG. 1B, the C-terminal loop of C-subunit may provide an area where all three PP2A subunits interact. In particular, the C-terminal loop may recognize a surface groove at the interface between the B-subunit and A-subunit.

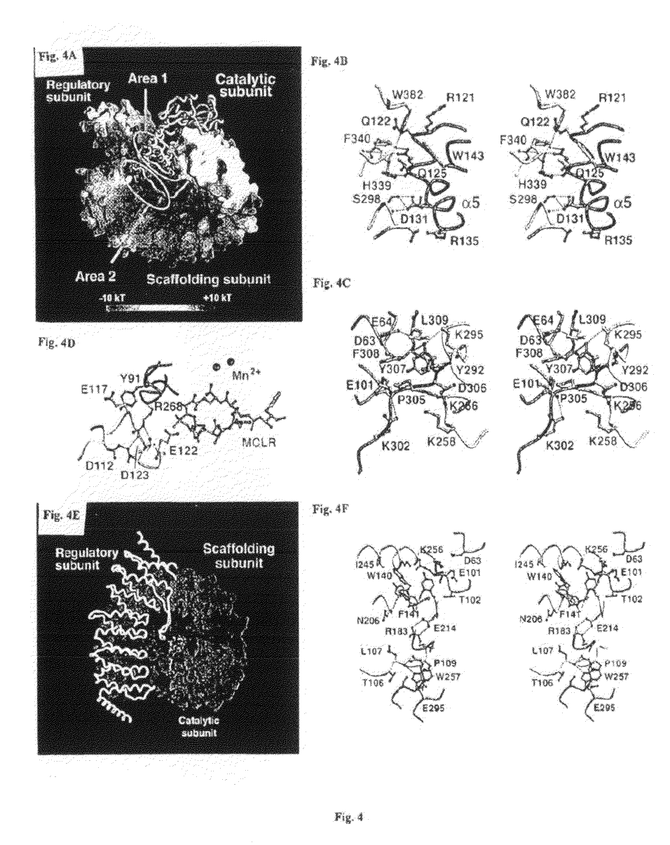

As indicated by the green circles in FIG. 4A, two areas make up the interface between the C-subunit (blue, wire diagram) and the B-subunit (surface representation with electrostatic potential) of PP2A holoenzyme. Area 1 includes amino acids from HEAT-like repeats 6-8 of B-subunit which appear to interact with the region surrounding helix α-5 of the C-subunit. FIG. 4B shows a portion of Area 1. with helix α-5 of the C-subunit shown in blue and HEAT-like repeal elements of the B-subunit shown in yellow. Interactions between the C-subunit and the B-subunit in this portion of the interface appear to be mediated mainly through, hydrogen bonding as indicated by the red dotted lines. Specifically, the polar side chain of Gln125 (Q125) of the C-subunit appears to make a pair of hydrogen bonds to the main chain atoms of B-subunit at the center of this interface. The aliphatic portion of the Gln 125 (Q125) side chain appears to mediate van der Waals contacts with His339 (H339), Phe340 (F340). and Trp382 (W382) of B-subunit. These interactions may be buttressed by three hydrogen bonds between the side chain of Asp131 (D131) of C-subunit and the amide nitrogen and side chain of Ser298 (S298) in B-subunit.

Area 2 includes the hydrophobic C-terminal loop of the C-subunit which appears to be nestled in a surface groove at the interface between A-subunit and B-subunit. FIG. 4C shows a portion of the interface included in Area 2 between the C-subunit (blue), the B-subunit (yellow) and A-subunit (green). The interface appears to include HEAT-like repeats 4 and 5 which are made up of amino acids 200-303 of B-subunit and appears to contain 7 amino acids that directly interact with A-subunit, including, for example, Glu214 (E214) which may hydrogen bond to Arg183 (R183) of the A-subunit. Additionally, numerous van der Waals contacts appear to be made at this interface. In particular, residues Pro305 (P305), Tyr307 (Y307), and Phe308 (F308) from C-subunit stack closely against each other and are surrounded by an aliphatic portion of the side chains of Lys256 (K256), Lys 258 (K258), Tyr292 (Y292) and Lys295 (K295) of the B-subunit and Asp63 (D63), Glu64 (E64) and Glu101 (E101) of A-subunit. These van der Waals interactions appear to be strengthened by six hydrogen bonds, one of which occurs between the side chain of Glu64 (E64) in A-subunit and the main chain amide nitrogen of Leu309 (L309) in C-subunit. It is of note that a mutation in Glu64 to Asp or Gly (E64D and E64G, respectively) in A-subunit has been observed in cancer cells, and these mutant proteins exhibited a significantly compromised ability to interact with the B-subunit. Without wishing to be bound by theory, this may suggest the importance of the interface at Area 2 for PP2A holoenzyme assembly and activity.

The two C-subunit/B-subunit binding regions appear to be conserved among B-subunits. For example, a Glu corresponding to Glu214 (E214) in B-subunit appears to be found in each subfamily of B-subunits. Therefore, without wishing to be bound by theory, at least a portion of the interactions between the A- and B-subunits may be conserved among the major families of regulatory subunits.

Western blot data suggests that Leu309 (L309) is fully methylated in the PP2A holoenzyme crystallized. However, the methyl group is not provided in FIG. 4, because the weak electron density in the data in this region. The binding groove in the B-subunit where the C-terminal loop of C-subunit appears to bind may be very acidic as illustrated in FIG. 4A. In this case, methylation of the C-terminus of the C-subunit may remove at least one negative charge which may facilitate, docking of the C-terminal loop of the C-subunit into this groove.

In addition to the interfaces at Area 1 and Area 2, an extended loop within HEAT-like repeat 2 of B-subunit may interact with C-subunit. FIG. 4D shows a portion of the extended loop of HEAT-like repeat 2 of B-subunit (yellow) interacting with the C-subunit (blue). As illustrated in FIG. 4D, Arg268 (R268) of the C-subunit appears to mediate van der Waals contact with MCLR (shown in cyan) and may form hydrogen bonds to two main chain carbonyl oxygen atoms of Asp119 (D119) and Glu122 (E122) and one to the side chain of Asp123 (D123) of the B-subunit.

FIG. 4E shows the convex surface of B-subunit (yellow, wire diagram) which appears to sit on the ridge of HEAT repeats 2-8 of the A-subunit (green surface representation). The interface between the B-subunit and the A-subunit is scattered into a large area and a small area. A portion of the large area including HEAT-like repeats 4 and 5 of B-subunit (yellow) and the ridge of HEAT repeats 2-5 of A-subunit (green) is shown in FIG. 4F. Trp140 (W140) and Phe141 (F141) of A-subunit appear to make multiple van der Waals interactions with hydrophobic residues in B-subunit, including, for example, Ile245, Lys249, and Tyr209. In addition, Arg183 (R183) of the A-subunit appears to donate a pair of charge-stabilized hydrogen bonds to Glu214 (E214) of the B-subunit, and Lys256 (K256) of B-subunit forms a salt bridge with three acidic residues in A-subunit, Asp63 (D63), Glu100 (E100) and Glu101 (E101). Trp257 (W257) of A-subunit also appears to form hydrogen bonds to main chain carbonyl oxygen of residue Leu107 (L107) of B-subunit in this region.

Referring again to FIG. 3, it is apparent that the residues in the B-subunit that appear to mediate contacts in the C-subunit and A-subunit, as represented by blue and green circles, respectively, may be highly conserved. For example, Glu214 (E214) which may accept two hydrogen bonds from Arg183 (R183) of A-subunit as illustrated in FIG. 4F, and Asp123 (D123) which may from hydrogen bonds to Arg268 (R268) of the C-subunit as illustrated in FIG. 4D appear to be conserved in B-subunit family members aligned across species. B-subunit residues that may contribute to hydrogen bonds at interfaces of Area 1, Lys295 (L295) and Ser298 (S298), and Area 2, His339 (H339), also appear to be conserved across members of the B-subunit family. Without wishing to be bound by theory, conservation among residues in the B-subunit that may mediate contacts with the A- or C-subunits may indicate that the binding interactions described above are conserved in PP2A holoenzymes across species.

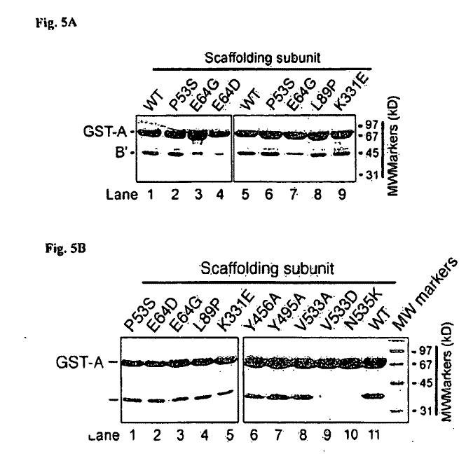

Mutations in the A-subunit may effect interactions with the B-subunit. For example, A-subunit mutants including, E64G, E64D, B53S, L89P, K331E, D492G, and V533A, show compromised binding to the B-subunit. As shown in FIG. 5A, mutations to Glu64 (E64) to Gly (E64G) or Asp (E64D) appear to compromise binding of the A-subunit to B-subunit which may be consistent with the observed interaction of Glu64 (E64) with various amino acids in the B-subunit. In contrast, FIG. 5A shows that mutations of Pro53 (P53S), Leu89 (L89P), and Lys331 (K331F) of A-subunit do not appear to affect binding to B-subunit and, therefore, may not be involved in interactions with B-subunit.

Mutations in the A-subunit may also affect interactions with the C-subunit. As indicated in FIG. 5B, mutations of residues outside of the ridge of HEAT repeats 11-15 which appear to form an interface with the C-subunit including, Pro53 (P53S), Glu64 (E64D and E64G), Leu 89 (L89P), and Lys331 (K331E) appear to have very little impact on interactions between the A-subunit and the C-subunit. However, C-subunit mutations Tyr456 (Y456A), Tyr495 (Y495A), and Val533 (V533A). which include residues thought to be at the interface between A- and C-subunits also do not appear to significantly weaken the interaction with C-subunit. Without wishing to be bound by theory, interactions between A-subunit and C-subunit appear to be extremely strong, so these interactions may be able to withstand the mild mutations while retaining effective binding. In contrast, the mutation of Val533 to a charged residue, Asp (V533D), appears to result in significant inhibition of A-subunit and C-subunit binding, and a mutation of Asn535 (N535K) which may mediate hydrogen bonding to Asn87 at the interface A-subunit and C-subunit also appears to inhibit interactions between the A-subunit and the C-subunit.

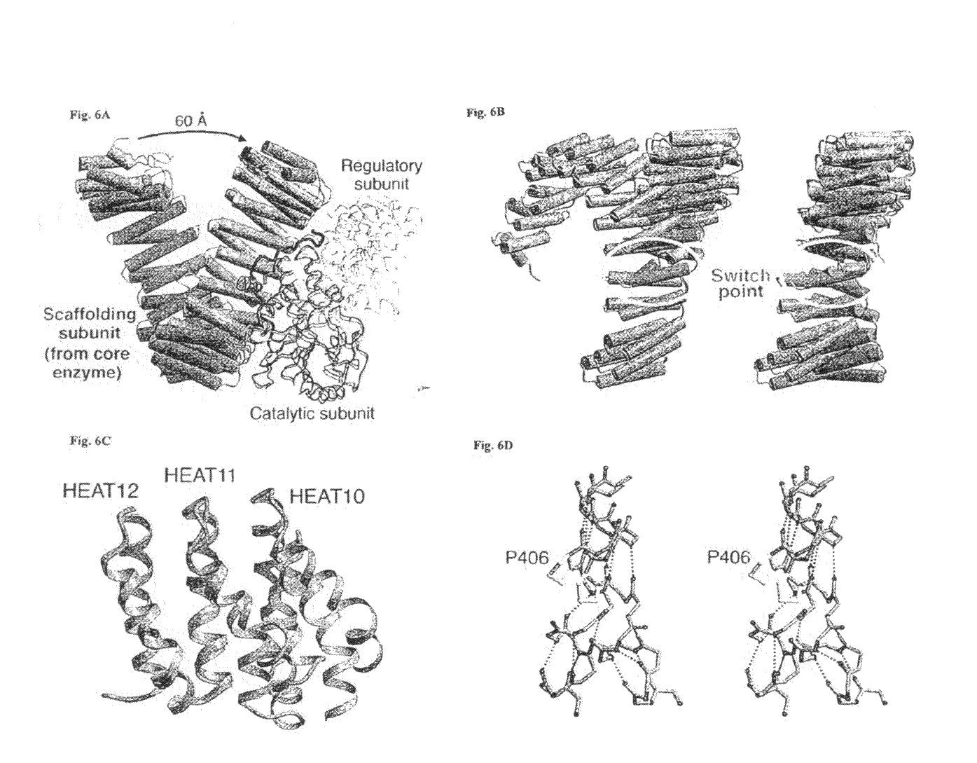

Conformational changes in the A-subunit have been noted as a result of binding to the C-subunit to form PP2A core as indicated in co-pending PCT Patent Application No. PCT/US2007/81260. Additional conformational changes appear to result from the binding of B-subunit to the PP2A core. For example, the overlay of PP2A core (C-subunit is orange and A-subunit is purple) and PP2A holoenzyme (G-subunit is blue, A-subunit is green and B′-subunit is yellow) shown in FIG. 6A appears to suggest a significant conformational change in the A-subunit as a result of B-subunit binding. Binding of B-subunit to PP2A core appears to force the N-terminal HEAT repeats of A-subunit to twist resulting in movement of the N-terminus of the A-subunit by as much as 50-60 Å. Additionally, the N-terminus and C-terminus of the A-subunit may be as much as 25 Å closer as a result of this conformational change. For example, the N- and C-termini of the A-subunit shown in FIG. 6A are about 50 Å apart in the PP2A holoenzyme as compared to about 70 Å in PR2A core.

An overlay of the A-subunit of PP2A core (purple) and PP2A holoenzyme (green) when the C-terminal 5 HEAT repeats are aligned and an a overlay of PP2A core (purple) and PP2A holoenzyme (green) when the N-terminal 10 HEAT repeats, FIG. 6B, left panel and FIG. 6B, right panel, respectively, indicates that a rearrangement of HEAT repeat 11 in the A-subunit as a result of B-subunit binding. As indicated in the overlay of HEAT repeats 10-12 from PP2A core (purple) and PP2A holoenzyme (green) shown in FIG. 6C, interactions both within HEAT repeat 11 and between HEAT repeats 11 and 10 or 12 may be altered as a result of B-subunit binding to PP2A core. For example, as illustrated in FIG. 4D, the distance between the carbonyl oxygen of Ser401 and the amide nitrogen of Leu405 appears to be about 3.0 Å in HEAT repeat 11 in PP2A core (purple) suggesting the presence of a hydrogen bond. In contrast, in PP2A holoenzyme (green), the distance between the carbonyl oxygen of Ser401 and the amide nitrogen of Leu405 appears to be about 4.7 Å which is beyond the range of a hydrogen bond. Thus, a hydrogen bond between residues Ser401 and Leu405 of HEAT repeat 11 may be broken contributing to a conformational rearrangement or the A-subunit as a result of B-subunit binding. PP2A core must associate with four different classes of the B-subunits and must act to remove phosphate groups on a variety of substrates. Without wishing to be bound by theory, the flexibility of the A-subunit may help facilitate binding to both B-subunits and substrate proteins.

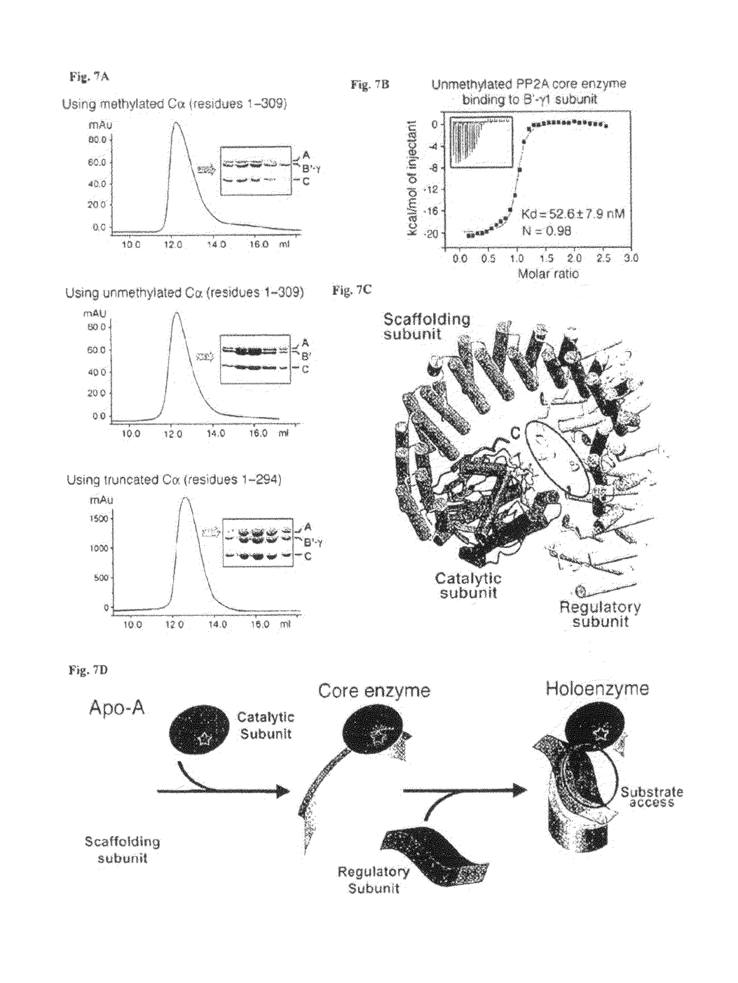

B-subunit has been shown to require methylation of the C-terminal Leu309 (L309) of the C-subunit of PP2A core for binding. However, the structural observation that Leu309 (L309) of the C-subunit is not structurally stable in PP2A holoenzyme crystals and, therefore, not visible in the structural data provided herein may provide evidence that the B-subunit in PP2A holoenzyme may bind unmethylated PP2A core. Additionally, PP2A cores formed by associating fully methylated C-subunit, unmethylated C-subunit, and a truncated C-subunit missing the C-terminal 15 amino acids (1-294) with A-subunit, each appear to form a stable PP2A holoenzymes as illustrated in FIG. 7A. Moreover, molecular models, obtained for PP2A holoenzyme containing either unmethylated C-subunit or truncated C-subunit (1-294) were prepared and refined to 3.8 Å and 3.6 Å resolution, respectively, (see Table 1 and FIG. 7B) and appear nearly identical to each other having an rmsd of about 0.6 Å for all backbone alpha carbon atoms when compared. Moreover, an rmsd of 0.66 Å over 1256 aligned alpha carbon atoms is obtained for truncated PP2A holoenzyme compared to fully methylated, native PP2A holoenzyme and the A-subunit/C-subunit interface and A-subunit/B-subunit interface appear similar in PP2A holoenzymes prepared with fully methylated C-subunit and truncated C-subunit indicating that the absence of methylation may not cause significant changes in the PP2A holoenzyme.

Various embodiments of the invention are directed to the atomic coordinates of PP2A holoenzyme and the use of these atomic coordinates to design or identify molecules that specifically inhibit or activate PP2A holoenzyme. For example, in one embodiment, the atomic coordinates of PP2A holoenzyme may be used to design and/or screen inhibitor molecules that bind to the PP2A C-subunit and interrupt binding of A-subunit or B-subunit. In another embodiments, the atomic coordinates of PP2A holoenzyme may be used to design and/or screen inhibitor molecules that bind to the A-subunit or B-subunit and, for example, inhibit the ability of the C-subunit of the PP2A core to bind a B-subunit to form the PP2A holoenzyme. In further embodiments, the atomic coordinates of PP2A holoenzyme may be used to design and/or screen molecules that inhibit the flexibility of the A-subunit or one or more B-subunit, such that C-, A- and B-subunits may not contact each other or a substrate protein cannot be brought into contact with the active site of the C-subunit. In still other embodiments, the atomic coordinates of PP2A holoenzyme may be used to design and/or screen activators of PP2A holoenzyme by, for example, increasing the affinity of the C-subunit for the A-subunit or inducing a bend in the A- or B-subunit that allows C-subunit to interact with these subunits more efficiently.

Embodiments encompassing the design and/or screening of molecules that inhibit PP2A holoenzyme activity may include inhibiting the activity PP2A C-subunit and/or inhibiting the ability of the PP2A C-subunit to bind to other components of PP2A core or PP2A holoenzyme. For example, in various embodiments, binding of an inhibitor molecule to the C-subunit may selectively reduce or eliminate the activity of PP2A holoenzyme by reducing the ability of the C-subunit to bind the A-subunit or the B-subunit by, for example, interrupting the binding interface between the C-subunit and the A-subunit or interrupting the binding interface between the C-subunit arid the B-subunit. In other embodiments, binding of an inhibitor molecule may reduce or eliminate modifications to the C-subunit, such as, for example, phosphorylation or methylation by inhibiting binding or activity of activating phosphorylases and/or methyl transferases. In additional embodiments, the atomic coordinates of PP2A holoenzyme described herein may be used to design and/or screen molecules that activate PP2A catalytic activity by, for example, stimulating activating phosphorylation and/or methylation or mimicking the binding of the A-subunit, B-subunit or a combination thereof to the C-subunit in the absence of indigenous A- or B-subunit.

Such inhibitors of the PP2A C-subunit may be designed or screened using any method known in the art. For example, in certain embodiments, the atomic coordinates of the PP2A C-subunit of PP2A holoenzyme may be identified, reconstituted and/or isolated in silico (i.e., using a computer processor, software, and a computer/user interface) and used to design or screen molecules that may fit within the interface wherein the C-subunit binds the A-subunit or one or more B-subunits. Compounds designed or identified using such methods may substantially mimic the shape, size, and/or charge of a portion of the A-subunit or a B-subunit and may bind to the C-subunit at the interface. For example, in one embodiment, a portion of the C-subunit encompassing the atomic coordinates of amino acids 122-135 and 143 and a portion of the B-subunit encompassing the atomic coordinates amino acids 297-298, 339-340, and 382 of B′-subunit may be used to design and/or screen compounds that substantially mimic the structural features of portions of the B-subunit and are substantially complementary to portions of the C-subunit. Such compounds may bind to the C-subunit and inhibit binding of the B-subunit or interrupt interactions between the C-subunit and the B-subunit thereby inhibiting PP2A holoenzyme activity. In other embodiments, portions of any of the interfaces described and illustrated in any of FIG. 4 or FIG. 6 may be used to design and/or screen compounds that may substantially mimic the shape, size, and/or charge of a portion of the A-subunit or a B-subunit and may bind to the C-subunit at the interface.

In some embodiments, a portion of the atomic coordinates defining the C-subunit of the PP2A holoenzyme encompassing a binding interface to an A-subunit or one or more B-subunits may be utilized to design and/or screen compounds that may inhibit PP2A holoenzyme activity. For example, a portion of the atomic coordinates of the C-subunit encompassing any of the interfaces described and illustrated, in FIG. 4 may be reconstituted and/or isolated in silico and used to identify compounds that substantially mimic a portion of the C-subunit and/or are substantially complementary to a portion, of the B-subunit at the interface Compounds identified in such embodiments may bind to one or more B-subunits and inhibit binding of the C-subunit or interrupt interactions at the interface between the B- and C-subunits thereby inhibiting activation of the C-subunit.

Other embodiments of the invention include molecules designed and screened to bind to the A-subunit and inhibit various aspects of A-subunit activity thereby inhibiting PP2A holoenzyme. For example, in one embodiment, an inhibitor may be designed or molecules may be screened and identified that binds to the A-subunit in a similar manner to the C-subunit or one or more B-subunits. For example, a molecule may be identified that binds to a portion of the A-subunit encompassing at least a portion of HEAT repeats 11-15. Similarly, in some embodiments, an inhibitor may be designed or molecules may be screened and identified that bind to a portion of the A-subunit at the A-subunit/B-subunit interface. For example, in one embodiment, a compound may be identified that binds to a portion of the A-subunit encompassing HEAT repeats 1-8. Molecules identified using such methods may interrupt or inhibit binding of the C-subunit or one or more B-subunits to the A subunit thereby inhibiting assembly of the PP2A holoenzyme.

In still other embodiments, an inhibitor may be designed or a molecule may screened and identified that inhibits or reduces the flexibility of the A-subunit thereby, for example, reducing or eliminating the ability of the A-subunit to bring the C-subunit and one or more B-subunits or other regulatory or substrate proteins into proximity, such that the PP2A holoenzyme may be activated. Embodiments including the design or screening of inhibitors which reduce flexibility of the A-subunit may include designing or screening any number of compounds which interact with the A-subunit in any number of ways. For example, in one embodiment, an inhibitor may be identified that binds between one or more HEAT repeats limiting the movement of these HEAT repeats. In another embodiment, a compound may be identified that binds to the A-subunit and various contacts made by the compound may reduce the ability of the A-subunit to flex. For example, a compound may bind between HEAT repeats on opposite ends of the A-subunit and inhibit A-subunit bending. In still another embodiment, a compound may be identified that interacts with one or more consecutive HEAT repeats reducing or eliminating the ability of the portion of the A-subunit encompassing these HEAT repeats to flex and reducing the overall flexibility of the A-subunit. In yet another embodiment, a compound may bind to one or more HEAT repeats and induce a bend in the A-subunit which may, for example, activate PP2A holoenzyme assembly or activate PP2A holoenzyme catalytic activity.

The invention provided herein above also encompasses inhibitors designed or identified that bind to the B-subunit. For example, a portion of the atomic coordinates of the B-subunit encompassing any of the interfaces described and illustrated in FIG. 4 may be reconstituted and/or isolated in silico and used to identify compounds that substantially mimic a portion of the C-subunit and/or are substantially complementary to a portion of the B-subunit at the interface. Compounds identified in such embodiments may bind to one or more B-subunits and inhibit binding of the C-subunit or interrupt interactions at the interface between the B- and C-subunits thereby inhibiting activation of the C-subunit. Inhibitors of a B-subunit may bind on either a convex or concave side of the B-subunit and may, therefore, interrupt interactions with either the C-subunit, the A-subunit or both.

In other embodiments, compounds may be identified or designed that bind to a portion of one or more B-subunits and reduce or inhibit flexibility of the B-subunit. For example, in one embodiment, ah inhibitor may be identified that binds between one of more HEAT-like repeats limiting the movement of these HEAT-like repeats. In another embodiment, a compound may be identified that binds to a B-subunit and various contacts made by the compound may reduce the ability of the B-subunit to flex. For example, a compound may bind between HEAT-like repeats on opposite ends of the B-subunit and inhibit B-subunit bending. In still another embodiment, a compound may be identified that interacts with one or more consecutive HEAT-like repeats reducing or eliminating the ability of the portion of a B-subunit encompassing these HEAT-like repeats to flex reducing the overall flexibility of the B-subunit. In yet another embodiment, a compound may bind to one or more HEAT-like repeals and induce a bend in the B-subunit which may, for example, activate PP2A holoenzyme assembly or activate PP2A holoenzyme catalytic activity.

In particular embodiments, the inhibitors may be identified or designed that bind to the concave side of one or more B-subunits as illustrated in FIG. 2A, top panel. Such inhibitors may bind to and inhibit the B-subunit by reducing the ability of the B-subunit to bind and identify substrate proteins or bring substrate proteins into contact with the active site of the C-subunit. For example, such an inhibitor may be substantially basic and may include a shape that is at least partially complementary to a portion of the concave side of the B-subunit. Such an inhibitor may also include one or more structural features associated with any number of substrate proteins. For example, substrate proteins may be aligned and structural features of portions of the substrate proteins having similarity, may be included as structural features in an inhibitor.

In any of the embodiments described above, a designed or identified inhibitor molecule may have a three-dimensional structure corresponding to at least a portion of PP2A holoenzyme. For example, an inhibitor may be identified by applying a three-dimensional modeling algorithm to the at least a portion of the atomic coordinates of the PP2A holoenzyme encompassing, for example, a region of the C-subunit where the inhibitor binds or a region of one or more subunits involved in an interface with an A-subunit, one or more B-subunits, substrate or regulatory protein and electronically screening stored spatial coordinates of candidate compounds against the atomic coordinates of the PP2A holoenzyme or a portion thereof. Candidate compounds that are identified as substantially complementary to the portion of the PP2A holoenzyme modeled, or designed to be substantially complementary to the portion of the PP2A holoenzyme modeled. Candidate compounds so identified may be synthesized using known techniques and then tested for the ability to bind to PP2A holoenzyme. A compound that is found to effectively bind the PP2A holoenzyme may be identified as an “inhibitor” of PP2A activity if it can then be shown that the binding of the compound reduces or inhibits the activity of the PP2A. Such “inhibitors” may then be used to modulate the activity of PP2A in vitro or in vivo. In still other embodiments, such “inhibitors” of PP2A may be administered to a subject or used as part of a pharmaceutical composition to be administered to individuals in need thereof.

The terms “complementary” or “substantially complementary” as used herein, refers to a compound having a size, shape, charge or any combination of these characteristics that allow the compound to substantially fill contours created by applying an three-dimensional modeling algorithm to a portion of the PP2A holoenzyme. A compound that substantially fills without overlapping portions of the various elements that make up the PP2A holoenzyme, even if various portions of the space remain unfilled, may be considered “substantially complementary”.

The terms “similar” or “substantially similar” may be used to describe a compound having a size, shape, charge or any combination of these characteristics similar to a compound known to bind PP2A holoenzyme. For example, an identified compound having a similar size, shape, and/or charge to a portion of the C-subunit may be considered “substantially similar” to the C-subunit.

Any inhibitor identified using the techniques described herein, may bind to PP2A with at least about the same affinity of the protein which binds at a selected interface or a known inhibitor to a known binding site, and in certain embodiments, the inhibitor may have an affinity for PP2A that is greater than the affinity of the natural or known substrate for PP2A. Thus, such inhibitors may bind to PP2A and inhibit the activity of PP2A, thereby providing methods and compounds for modulating the activity of PP2A. Without wishing to be bound by theory, inhibition of PP2A may reduce or PP2A mediated serine/threonine phosphorylation, and modulating the activity of PP2A may provide the basis for treatment of various cell cycle modulation or proliferative disorders including, for example, cancer and autoimmune disease.

Determination of the atomic coordinates of any portion of the PP2A holoenzyme may be carried out by any method known in the art. For example, the atomic coordinates provided in embodiments of the invention, or the atomic coordinates provided by other PP2A crystallographic or NMR structures including, but not limited to, crystallographic or NMR data for PP2A core, PP2A holoenzyme or individual A, B or C components of PP2A, may be provided to a molecular modeling program and the various portions of PP2A holoenzyme described above may be visualized. In other embodiments, two or more sets of atomic coordinates corresponding to various portions of PP2A holoenzyme may be compared and composite coordinates representing the average of these coordinates may be used to model the structural features of the portion of PP2A holoenzyme under study. The atomic coordinates used in such embodiments may be derived from purified PP2A holoenzyme, individual A, B or C subunits, or PP2A bound to other regulatory proteins, substrate proteins, accessory proteins, protein fragments or peptides. In general, atomic coordinates defining a three-dimensional structure of a crystal of a PP2A holoenzyme that diffracts X-rays for the determination of atomic coordinates to a resolution of 5 Angstroms or better may be preferred.

Having defined the structural features of PP2A holoenzyme, mimetics or small molecules substantially complementary to various portions of the PP2A holoenzyme, such as those described above, may be designed. Various methods for molecular design are known in the art, and any of these may be used in embodiments of the invention. For example, in some embodiments, compounds may be specifically designed to fill contours of a portion of the PP2A holoenzyme at the interfaces between PP2A. holoenzyme subunits or in portions of the PP2A holoenzyme where other factors or substrate proteins interact. In other embodiments, random compounds may be generated and compared to the spatial coordinates such as a portion of PP2A holoenzyme. In still other embodiments, stored spatial coordinates of candidate compounds contained within a database may be compared to the spatial coordinates of a portion of PP2A holoenzyme. In certain embodiments, molecular design may be carried out in combination with molecular modeling.

In particular embodiments, the atomic coordinates of a subunit bound to another subunit of PP2A holoenzyme or another factor bound to a portion of PP2A holoenzyme, as provided herein, may be used as a basis for mimetic or small molecule inhibitor design or identification. In such embodiments, compounds that mimic the structure of a compound bound to PP2A holoenzyme and maintain the molecular contacts, such as, for example, hydrogen bonds and van der Waals contacts, may be created or identified. Such compounds may bind PP2A holoenzyme and/or inhibit PP2A activity. In some embodiments, additional features may be added to a compound or portion of a subunit's backbone to create a new compound which provides improved contact, between the PP2A holoenzyme and a compound. For example, in one embodiment, a compound may include, an additional atom that brings a portion of the compound into closer proximity to a moiety on a portion of PP2A holoenzyme, thereby improving van der Waals interaction or hydrogen bonding potential. In another embodiment, a compound may contain an atom or group of atoms that provide one or more additional hydrogen bonds or one or more additional van der Waals contacts.

Methods for performing structural comparisons of atomic coordinates of molecules including those derived from protein crystallography are well known in the art, and any such method may be used in various embodiments to test candidate PP2A holoenzyme binding compounds for the ability to bind a portion of PP2A holoenzyme. In such embodiments, atomic coordinates of designed, random or stored candidate compounds may be compared against a portion of the PP2A holoenzyme structure or the atomic coordinates of a compound bound to PP2A holoenzyme. In other such embodiments, a designed, random or stored candidate compound may be brought into contact with a surface of the PP2A holoenzyme, and simulated hydrogen bonding and/or van der Waals interactions may be used to evaluate or test the ability of the candidate compound to bind the surface of PP2A holoenzyme. Structural comparisons, such as those described in the preceding embodiments may be carried out using any method, such as, for example, a distance alignment matrix (DALI), Sequential Structure Alignment Program (SSAP), combinatorial extension (CE) or any such structural comparison algorithm. Compounds that appear to mimic a portion of the PP2A holoenzyme structure under study or a compound known to bind PP2A holoenzyme, such as, for example, a substrate protein, or that are substantially complementary and have a likelihood of forming sufficient interactions to bind to PP2A holoenzyme may be identified as a potential PP2A holoenzyme binding compound.

In some embodiments, compounds identified as described above may conform to a set of predetermined variables. For example, in one embodiment, the atomic coordinates of an identified PP2A holoenzyme binding compound when compared with a native PP2A holoenzyme binding compound or a subunit of PP2A using one or more of the above structural comparison methods may deviate from an rmsd of less than about 10 angstroms. In another embodiment, the atomic coordinates of the compound may deviate from the atomic coordinates of PP2A holoenzyme by less than about 2 angstroms. In still another embodiment, the identified PP2A holoenzyme binding compound may include one or more specific structural features known to exist in a native PP2A holoenzyme binding compound or a subunit of PP2A holoenzyme, such as, for example, a surface area, shape, charge distribution over the entire compound or a portion of the identified compound.

Compounds identified by the various methods embodied herein may be synthesized by any method known in the art. For example, identified compounds may be synthesized using manual techniques or by automation using in vitro methods such as, various solid state or liquid state synthesis methods. Direct peptide synthesis using solid-phase techniques is well known and utilized in the art (see, e.g., Stewart et al., Solid-Phase Peptide Synthesis, W. H. Freeman Co., San Francisco, Calif. (1969); Merrifield, J. Am. Chem. Soc., 85:2149-2154 (1963)). Automated synthesis may be accomplished, for example, using an Peptide Synthesizer using manufacturer's instructions. Additionally, in some embodiments, one or more portion of the PP2A modulators described herein may be synthesized separately and combined using chemical or enzymatic methods to produce a full length modulator.

Compounds identified using various methods of embodiments of the invention may be further tested for binding to PP2A holoenzyme and/or to determine the compound's ability to inhibit activity of PP2A holoenzyme or modulate the activity of PP2A holoenzyme by, for example, testing for pTyr activity or testing the candidate compound for binding to PP2A holoenzyme. Such testing may be carried out by any method. For example, such methods may include contacting a known substrate with an identified compound and detecting binding to PP2A by a change in fluorescence in a marker or by detecting the presence of the bound compound by isolating the PP2A/candidate compound complex and testing for the presence of the compound. In other embodiments, PP2A activity may be tested by, for example, isolating a substrate peptide that has or has not been phosphorylated by PP2A or isolating a PP2A holoenzyme that has been contacted with the candidate compound. Such methods are well known in the art and may be carried out in vitro, in a cell-free assay, or in vivo, in a cell-culture assay.

Embodiments of the invention also include pharmaceutical compositions including inhibitors that bind PP2A and inhibit PP2A activity or compounds that are identified using methods of embodiments described herein above and a pharmaceutically acceptable carrier or excipient. Such pharmaceutical compositions may be administered to an individual in an effective amount to alleviate conditions associated with PP2A activity.

Various embodiments of the invention also include a system for identifying a PP2A modulator. Such systems may include a processor and a computer readable medium in contact with the processor. The computer readable medium of such embodiments may at least contain the atomic coordinates of PP2A holoenzyme. In some embodiments, the computer readable medium may further contain one or more programming instructions for comparing at least a portion of the atomic coordinates of the PP2A holoenzyme with atomic coordinates of candidate compounds included in a library of compounds. In other embodiments, the computer readable medium may further contain one or more programming instructions for designing a compound that mimics at least a portion of the PP2A holoenzyme or that is substantially complementary to a portion of the PP2A holoenzyme. In still other embodiments, the computer readable medium may contain one or more programming instructions for identifying candidate compounds or designing a compound that mimics a portion of PP2A holoenzyme within one or more user defined parameters. For example, in some embodiments, a compound may include a charged molecule at a particular position corresponding to one or more positions within the atomic coordinates of PP2A holoenzyme, and in other embodiments, the compound may deviate from the carbon backbone or surface model representation of PP2A holoenzyme by, for example, an rmsd of less than about 10 Å. In still other embodiments, a user may determine the size of a candidate compound or the portion of the PP2A holoenzyme that is utilized in identifying mimetic candidate compounds. Further embodiments may include one or more programming instructions for simulating binding of an identified candidate compound to PP2A holoenzyme or a portion of the PP2A holoenzyme. Such embodiments may be carried out using any method known in the art, and may provide an additional in silico method for testing identified candidate compounds.

The invention described herein encompasses pharmaceutical compositions including a therapeutically effective amount of an inhibitor in dosage form and a pharmaceutically acceptable carrier, wherein the compound inhibits the phosphotyrosyl or phosphoserosyl activity of PP2A. In another embodiment, such compositions include a therapeutically effective amount of an inhibitor in dosage form and a pharmaceutically acceptable carrier in combination with a chemotherapeutic and/or radiotherapy, wherein the inhibitor inhibits the phosphotyrosyl or phosphoserosyl activity of PP2A, promoting apoptosis and enhancing the effectiveness Of the chemotherapeutic and/or radiotherapy. In various embodiments of the invention, a therapeutic composition for modulating PP2A activity can be a therapeutically effective amount of a PP2A inhibitor.

Embodiments of the invention also include methods for treating a patient having a condition characterized by aberrant cell growth, wherein administration of a therapeutically effective amount of a PP2A inhibitor is administered to the patient, and the inhibitor binds to PP2A inducing apoptosis within the area of the patient exhibiting aberrant cell growth. The method may further include the concurrent administration of a chemotherapeutic agent, such as, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

The PP2A inhibitors of the invention may be administered in an effective amount. An “effective amount” is an amount of a preparation that alone, or together with further doses, produces the desired response. This may involve only slowing the progression of the disease temporarily, although it may involve halting the progression of the disease permanently or delaying the onset of or preventing the disease or condition from occurring. This can be monitored by routine methods known and practiced in the art. Generally, doses of active compounds may be from about 0.01 mg/kg per day to about 1000 mg/kg per day, and in some embodiments, the dosage may be from 50-500 mg/kg. In various embodiments, the compounds of the invention may be administered intravenously, intramuscularly, or intradermally, and in one or several administrations per day. The administration of inhibitors can occur simultaneous with, subsequent to, or prior to chemotherapy or radiation.

In general, routine experimentation in clinical trials will determine specific ranges for optimal therapeutic effect for each therapeutic agent and each administrative protocol and administration to specific patients will be adjusted to within effective and safe ranges depending on the patient's condition and responsiveness to initial administrations. However, the ultimate administration protocol will be regulated according to the judgment of the attending clinician considering such factors as age, condition and size of the patient, the potency of the PP2A inhibitor administered, the duration of the treatment and the severity of the disease being treated. For example, a dosage regimen of a PP2A inhibitor to reduce cellular proliferation or induce apoptosis can be oral administration of from about 1 mg to about 2000 mg/day, preferably about 1 to about 1000 mg/day, more preferably about 50 to about 600 mg/day, in two to four divided doses. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient's tolerance permits. Multiple doses per day are contemplated to achieve appropriate systemic levels of compounds. Generally, a maximum dose is used, that is, the highest safe dose according to sound medical judgment. However, an individual patient may insist upon a lower dose or tolerable dose for medical reasons, psychological reasons or for virtually any other reason.

Embodiments of the invention also include a method of treating a patient with cancer or an autoimmune disease by promoting apoptosis, wherein administration of a therapeutically effective amount of one or more PP2A inhibitors, and the PP2A inhibitor inhibit the phosphotyrosyl or phosphoserosyl activity of PP2A. The method may further include concurrent administration of a chemotherapeutic agent including, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

A variety of administration routes are available. The particular mode selected will depend upon the severity of the condition being treated and the dosage required for therapeutic efficacy. The methods of the invention may be practiced using any mode of administration that is medically acceptable, meaning any mode that produces effective levels of active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes may be particularly suitable for purposes of the present invention.

In one aspect of the invention, a PP2A inhibitor as described herein, with or without additional biological or chemotherapeutic agents or radiotherapy, does not adversely affect normal tissues while sensitizing aberrantly dividing cells to the additional chemotherapeutic/radiation protocols. While not wishing to be bound by theory because the PP2A inhibitors specifically target PP2A, marked and adverse side effects may be minimized. In certain embodiments, the composition or method may be designed to allow sensitization of the cell to chemotherapeutic agents or radiation therapy by administering the ATPase inhibitor prior to chemotherapeutic or radiation therapy.

The term “pharmaceutically-acceptable carrier” as used herein, means one or more compatible solid or liquid fillers, diluents or encapsulating substances which are suitable for administration into a human. The term “carrier” or “excipient” denotes an organic or inorganic ingredient, natural or synthetic, with which the active ingredient is combined to facilitate the application. The components of the pharmaceutical compositions are also capable of being co-mingled with the molecules of the present invention and with each other, in a manner such that there is no interaction which would substantially impair the desired pharmaceutical efficacy.

The delivery systems of the invention are designed to include time-released, delayed release or sustained release delivery systems such that the delivery of the PP2A inhibitors occurs prior to, and with sufficient time, to cause sensitization of the site to be treated. A PP2A inhibitor may be used in conjunction with radiation and/or additional anti-cancer chemical agents. Such systems can avoid repeated administrations of the PP2A inhibitor compound, increasing convenience to the subject and the physician, and may be particularly suitable for certain compositions of the present invention.

Many types of release delivery systems are available and known to those of ordinary skill in the art including, but not limited to, polymer base systems, such as, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems including, for example: lipids including sterols, such as cholesterol, cholesterol esters and fatty acids or neutral fats, such as mono-, di- and tri-glycerides; hydrogel release systems; sylastic systems; peptide based systems; wax coatings; compressed tablets using conventional binders and excipients; partially fused implants; and the like. Specific examples include, but are not limited to: erosional systems in which the active compound is contained in a form within a matrix such as those described in U.S. Pat. Nos. 4,452,775, 4,667,014, 4,748,034, and 5,239,660 and diffusional systems in which an active component permeates at a controlled rate from a polymer, such as described in U.S. Pat. Nos. 3,832,253, and 3,854,480. In addition, pump-based hardware delivery systems can be used, some of which are adapted for implantation.

Use of a long-term sustained release implant may be desirable. Long-term release is used herein, and means that the implant is constructed and arranged to deliver therapeutic levels of the active ingredient for at least about 30 days, and preferably about 60 days. Long-term sustained release implants are well-known to those of ordinary skill in the art and include some of the release systems described above.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions may be prepared by uniformly and intimately bringing the active compound into association with a liquid carrier, a finely divided solid carrier, or both and then, if necessary, shaping the product.

Compositions suitable for parenteral administration conveniently include a sterile aqueous preparation of an ATPase inhibitor which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile fixed oils are conventionally employed as a solvent, or suspending medium. For this purpose, any bland fixed oil may be employed including synthetic mono- or di-glycerides. In addition, fatty acids, such as oleic acid, may be used in the preparation of injectables. Carrier formulation suitable for oral, subcutaneous, intravenous, intramuscular, etc. administrations can be found, for example, in Remington's Pharmaceutical Sciences, Mack Publishing Co., Easton, Pa. which is incorporated herein in its entirety by reference thereto.

### EXPERIMENTAL PROCEDURES

### Protein Preparation and Assembly of PP2A Holoenzyme

All constructs and point mutations were generated using a standard PCR-based cloning strategy. Full-length human PP2A A-subunit α (1-589), all mutants, and the regulatory B′-subunit were cloned into pGEX-2T vector (GE Healthcare) and overexpressed in E. coli strain BL21(DE3). The soluble fraction of the E. coli cell lysate was purified using glutathione resin (Qiagen) and fractionated by ion-exchange chromatography (Source 15Q, Amersham). Full-length human PP2A C-subunit α (1-309) was cloned into the baculovirus transfer vector pVL1392 (Pharmingen) as an N-terminal 8xHis-tagged protein. Recombinant baculovirus was generated using the BaculoGold co-transfection kit (Pharmingen). C-subunit was over-expressed in baculovirus-infected Hi-5 suspension culture arid purified to homogeneity on a Ni-NTA column (Qiagen) and fractionated by ion-exchange (Source 15Q, Amersham).

PP2A core was assembled by passing purified C-subunit, which was pre-incubated with an excess amount of MCLR or OA, through a stoichiometric amount of GST-A-subunit immobilized on glutathione resin. Assembled PP2A core was released by on-column thrombin cleavage and further purified by ion-exchange chromatography. Phosphatase assays were performed to ensure that there was no remaining activity for the PP2A core bound to the glutathione resin.

The PP2A core enzyme, if methylated, was methylated by a PP2A-specific leucine carboxyl methyltransferase (LCMT) in the presence of S-adenosyl methionine (SAM). LCMT and PP2A core enzyme, at a 1:2 molar ratio, was incubated on ice. Methylation was initiated by addition of SAM (S-adenosyl methionine) to a final concentration of 0.75 mM. The reaction was carried out at 22° C. and reached completion after 2-3 hours. The methylated PP2A core enzyme was purified away from LCMT by anion exchange chromatography, and extent of methylation was examined using an antibody that only recognizes the unmethylated C-terminus of C-subunit.

Following methylation, methylated PP2A core enzyme was incubated with a stoichiometric amount of B′-subunit. The PP2A holoenzyme was purified to homogeneity by gel filtration chromatography. In addition, the purified full-length C-subunit was used to generate a carboxy-terminally truncated variant (residues 1-294) through trypsin digestion. The truncated C-subunit was in turn used to form a core enzyme with A-subunit, which was then assembled into additional PP2A holoenzyme hetero-trimeric complexes, one involving B′-γ1 and the other involving B′-γ3. To facilitate structure determination, three PP2A holoenzyme complexes using seleno-methionine-substituted A-subunit and B′-γ1 subunit were prepared.

### Crystallization and Data Collection

Diffracting crystals were obtained for the three PP2A holoenzyme complexes described above, which were individually incubated with 1.2 molar equivalence of microcystin-LR (MCLR) prior to crystallization. Crystals were grown by hanging-drop vapor-diffusion by mixing the protein (˜8 mg/ml) with an equal volume of reservoir solution containing 10-15% PEG-8000, 0.1 M Tris-Cl pH 8.5, and 0.2 M magnesium sulfate. Small crystals appeared within a few hours. Macroseeding was used to generate single, large crystals. The crystals belong to the space group P212121, with a=109.29 Å, b=159.05 Å, and c=269.17 Å. There are two complexes per asymmetric unit, and the solvent content is approximately 80%. Crystals were slowly equilibrated in a cryoprotectant buffer containing reservoir buffer plus 20% glycerol (v/v) and were flash frozen in a cold nitrogen stream at −170° C. Native and selenium SAD data sets were collected at NSLS beamline X29 and processed using the software Denzo and Scalepack.

### Structure Determination

The structure of PP2A holoenzyme was determined by molecular replacement using three models: C-subunit and two fragments of the A-subunit (residues 9-415 and 416-589) from the structure of PP2A core provided in PCT Patent Application No. PCT/US2007/81260 (accession code 21E3). Fragments were located using the program PHASER. The backbone of B′-subunit was built into model-phased two-fold-averaged 2 Fo-Fc and Fo-Fc electron density maps. Side-chain interpretation was guided by a model-phased anomalous difference Fourier of SeMet SAD data collected from complexes containing SeMet-labeled A-subunit and B′-subunit components. Model building was performed using O, and the model was refined using CNS. Tight NCS restraints between the two complexes (50 kcal/mol) were applied throughout most of the refinement and these restraints were relaxed in the final cycles. Positional refinement was performed against maximum likelihood target with NCS-restrained and geometrically-restrained individual B-factor refinement, with weights adjusted on the basis of R-free. In tests, this refinement method consistently gave a lower R-free than, grouped B-factor refinement (which cannot be restrained by geometry in CNS) and was considerably better than adopting a single overall or per-domain B-factor model. The final atomic, model of the methylated holoenzyme has been refined to 3.3 Å resolution. For the methylated holoenzyme, the atomic model contains amino acids 2-309 for the C-subunit, residues 8-589 for A-subunit, and residues 38Δand 68-425 for B′-subunit. There is no electron density for residues 1-37 and 426-439 of B′-subunit which may reflect disorder in the crystals in these regions; The methyl group on the methylated carboxy-terminus of Cα was not modeled, because there is no clear electron density for this group at this resolution.

Due to nearly identical unit cells, structures of the two PP2A holoenzyme complexes containing unmethylated and truncated C-subunit were directly refined at 3.6 Å and 3.8 Å resolution. Similar positional and B-factor refinement protocols were used as described above. For PP2A holoenzyme complexes containing unmethylated C-subunit, truncated C-subunit (residues 1-294), B′-γ3 and B′-γ1, the atomic models contain amino acids 2-294 for C-subunit, residues 8-589 for A-subunit, and residues 38-66 and 68-425 for B′-γ3 or B′-γ1.

### GST-Mediated Pull-Down Assay

Approximately 30 μg of GST-A-subunit was bound to 30 μl of glutathione resin. The resin was washed with 200 μl assay buffer for three times to remove excess unbound Aα. Then 20 μg of WT or mutant B′-subunit was allowed to bind the resin in a 125-μl volume. After washing four times with an assay buffer containing 25 mM Tris, pH 8.0, 150 mM NaCl, and 2 mM dithiothreitol (DTT), remaining protein and resin were mixed with 15-μl SDS sample buffer and applied to SDS-PAGE. The results were visualized by SDS-polyacrylamide gel electrophoresis (SDS-PAGE) with Coomassie-blue staining.

What is claimed is:

1. A method for preparing a PP2A modulating compound comprising:
applying a three-dimensional molecular modeling algorithm to the atomic coordinates of at least a portion of PP2A holoenzyme;
determining spatial coordinates of the at least a portion of PP2A holoenzyme;
electronically screening stored spatial coordinates of candidate compounds against the spatial coordinates of the at least a portion of PP2A holoenzyme;
identifying a compound that is substantially similar to the at least a portion of PP2A holoenzyme; and
synthesizing the identified compound.

2. The method of claim 1, further comprising identifying a candidate compound that deviates from the atomic coordinates of the at least a portion of PP2A holoenzyme by a root mean square deviation of less than about 10 angstroms.

3. The method of claim 1, further comprising testing the identified compound for binding at least a portion of PP2A.

4. The method of claim 1, further comprising testing the identified compound for inhibiting PP2A activity.

5. The method of claim 1, further comprising testing the identified compound inhibits tyrosine phosphorylation, serine phosphorylation, threonine phosphorylation or a combination thereof catalyzed by PP2A holoenzyme.

6. The method of claim 1, wherein the step of electronically screening stored spatial coordinates further comprises identifying a compound that has a shape, a charge distribution, a size or a combination thereof substantially similar to a portion of PP2A holoenzyme.

7. The method of claim 1, wherein the at least a portion of the PP2A holoenzyme comprises an interface between any one of: scaffolding (A) subunit and catalytic (C) subunit, scaffolding (A) subunit and regulatory (B) subunit and regulatory (B) subunit and catalytic (C) subunit.

8. The method of claim 7, wherein the identified compound interrupts the interface and inhibits PP2A holoenzyme assembly.

9. The method of claim 1, wherein the identified compound binds PP2A holoenzyme.

10. A method for preparing a PP2A inhibitor comprising:
applying a three-dimensional molecular modeling algorithm to the atomic coordinates of PP2A holoenzyme;
determining spatial coordinates of a portion of PP2A holoenzyme corresponding to a concave surface of a regulatory (B) subunit;
electronically screening stored spatial coordinates of candidate compounds against the spatial coordinates of the at least a portion of PP2A holoenzyme corresponding to the concave surface of the regulatory (B) subunit;
identifying a compound that is substantially complementary to the concave surface of PP2A holoenzyme regulatory (B) subunit; and
synthesizing the identified compound.

11. The method of claim 10, further comprising identifying a compound that has a shape, a charge distribution, a size or a combination thereof substantially complementary to the concave surface of PP2A holoenzyme regulatory (B) subunit.

12. The method of claim 10, wherein the identified compound comprises a plurality of basic moieties.

13. The method of claim 10, wherein the identified compound inhibits entry of substrate into an active site of PP2A catalytic (C) subunit.

14. The method of claim 10, further comprising:
identifying one or more PP2A substrate proteins;
isolating at least a portion of the one or more PP2A substrate proteins where PP2A holoenzyme is likely to bind the one or more PP2A substrate proteins;
determining spatial coordinates of the at least a portion of the one or more PP2A substrate proteins; and
identifying a compound that is substantially similar to the at least a portion of the one or more PP2A substrate proteins.

15. The method of claim 14, wherein the step of isolating one or more PP2A substrate proteins further comprises:
identifying more than one PP2A substrate proteins;
performing an alignment of the more than one PP2A substrate proteins; and
isolating at least a portion of the more than one PP2A substrate proteins that share sequence similarity or secondary structure similarity.

16. The method of claim 10, further comprising testing the identified compound for binding to PP2A holoenzyme.

17. A pharmaceutical composition comprising:
an effective amount of a compound having a three-dimensional structure corresponding to atomic coordinates of at least a portion of PP2A; and
a pharmaceutically acceptable excipient or carrier.

18. The pharmaceutical composition of claim 17, wherein the compound binds to PP2A holoenzyme.

19. A system for identifying PP2A modulators comprising:
a processor; and
a processor readable storage medium in communication with the processor readable storage medium comprising the atomic coordinates of at least a portion of PP2A holoenzyme.

20. The system of claim 19, wherein the processor readable storage medium further comprises one or more programming instructions for:
applying a three-dimensional modeling algorithm to the atomic coordinates of PP2A holoenzyme;
determining spatial coordinates of at least a portion of the PP2A holoenzyme;
electronically screening spatial coordinates of candidate compounds with the spatial coordinates of the at least a portion of the PP2A holoenzyme; and
identifying a candidate compound whose spatial coordinates are substantially similar to the spatial coordinates of the at least a portion of the PP2A holoenzyme; or
identifying a candidate compound whose spatial coordinates are substantially complementary to the spatial coordinates of the at least a portion of the PP2A holoenzyme.

21. The system of claim 20, wherein the one or more programming instructions for identifying a candidate compound whose spatial coordinates are substantially similar to the spatial coordinates of the at least a portion of the PP2A holoenzyme comprise one or more programming instructions for identifying a compound that deviates from the spatial coordinates of the at least a portion of the PP2A holoenzyme by a user defined threshold.

22. The system of claim 20, wherein the one or more programming instructions for identifying a compound whose spatial coordinates are substantially similar to the at least a portion of the PP2A holoenzyme comprise one or more programming instructions for identifying a compound having one or more of:
a size within a user defined threshold;
a charge within a user defined threshold; or
a shape with a user defined threshold.

23. The system of claim 20, wherein the one or more programming instructions for electronically screening spatial coordinates of a candidate compound comprises one or more programming instructions for simulating binding of the candidate compound to the PP2A holoenzyme.

24. The system of claim 19, further comprising an output device in communication with the processor

25. The system of claim 24, wherein the processor readable storage medium further comprises one or more programming instructions for:
applying a three-dimensional modeling algorithm to the atomic coordinates of PP2A holoenzyme;
determining spatial coordinates of at least a portion of the PP2A holoenzyme;
generating a visual signal arid relaying the visual signal to the output device; and
electronically designing a compound that is substantially similar to the at least a portion of the PP2A holoenzyme; or
electronically designing a compound that is substantially complementary to the at least a portion of the PP2A holoenzyme.

26. A protein phosphatase 2A (PP2A) binding compound comprising a molecule having a three-dimensional structure corresponding to atomic coordinates derived from at least a portion of an atomic model of protein phosphatase 2A (PP2A) holoenzyme or protein phosphatase 2A (PP2A) holoenzyme bound to microcystin-LR.

27. The compound of claim 26, wherein the molecule is an inhibitor of protein phosphatase 2A (PP2A).

28. The compound of claim 26, wherein the molecule has a three-dimensional structure corresponding to atomic coordinates of at least a portion microcystin-LR or a combination thereof bound to protein phosphatase 2A (PP2A) holoenzyme; and
wherein the compound makes interactions with the catalytic (C) subunit of protein phosphatase 2A (PP2A) holoenzyme that correspond to at least a portion of the interactions observed between the catalytic (C) subunit of protein phosphatase 2A (PP2A) holoenzyme and microcystin-LR.

29. The compound of claim 28, wherein the molecule binds protein phosphatase 2A (PP2A) at a binding site for microcystin-LR on the catalytic (C) subunit of PP2A.

30. The compound of claim 26, wherein the molecule has a shape, a charge, a size or combinations thereof substantially corresponding to a portion of protein phosphatase 2A (PP2A) holoenzyme.

31. The compound of claim 30, wherein the molecule binds to a catalytic (C) subunit of protein phosphatase 2A (PP2A), a scaffolding (A) subunit of protein phosphatase 2A (PP2A) or a regulatory (B) subunit of protein phosphatase 2A (PP2A) at an interface between the catalytic (C) subunit and the scaffolding (A) subunit, a scaffolding (A) subunit and a regulatory (B) subunit, a catalytic (C) subunit and regulatory (B) subunit, or a combination thereof.

32. The compound of claim 26, wherein the molecule has a shape, a charge, a size or combinations thereof substantially complementary to a portion of protein phosphatase 2A (PP2A) holoenzyme.

33. The compound of claim 32, wherein the molecule is substantially complementary to a portion of a scaffolding (A) subunit of protein phosphatase 2A (PP2A) holoenzyme.

34. The compound of claim 33, wherein the molecule binds a scaffolding (A) subunit of PP2A holoenzyme and inhibits flexibility of the scaffolding (A) subunit.

35. The compound of claim 32, wherein the molecule is substantially complementary to a portion of a regulatory (B) subunit of protein phosphatase 2A (PP2A) holoenzyme.

36. The compound of claim 35, wherein the molecule inhibits access of substrate to the active site of protein phosphatase 2A (PP2A) holoenzyme

37. The compound of claim 35, wherein the molecule inhibits formation of an active protein phosphatase 2A holoenzyme.

38. The compound of claim 26, wherein the molecule binds to at least a portion of protein phosphatase 2A (PP2A) holoenzyme with a greater affinity than a naturally occurring substrate.

39. The compound of claim 26, wherein the molecule inhibits protein phosphatase 2A (PP2A) catalyzed tyrosine phosphorylation, serine phosphorylation, threonine phosphorylation or a combination thereof.

40. The compound of claim 26, further comprising a pharmaceutically acceptable excipient or carrier.

41. The compound of claim 26, wherein the molecule deviates from the atomic coordinates of the at least a portion of PP2A holoenzyme by a root mean square deviation of less than about 10 angstroms.

42. The compound of claim 26, wherein the molecule deviates from the atomic coordinates of the at least a portion of PP2A holoenzyme by a root mean square deviation of less than about 2 angstroms.
